(19) United States
(12) Patent Application Publication
(10) Pub. No.: US 2022/0280585 A1
(43) Pub. Date: Sep. 8, 2022

(54) PHARMACEUTICAL COMPOSITION CONTAINING CHAMPIGNON EXTRACT AND PEPPERMINT AND METHODS OF USE THEREOF

(71) Applicant: Poopermints, LLC, Boulder Junction, WI (US)

(72) Inventor: Paul Kuehn, Boulder Junction, WI (US)

(21) Appl. No.: 17/349,394

(22) Filed: Jun. 16, 2021

Related U.S. Application Data
(60) Provisional application No. 63/197,558, filed on Jun. 7, 2021.
(60) Provisional application No. 63/157,035, filed on Mar. 5, 2021.

(51) Int. Cl. A61K 36/534; A61K 36/07; A61K 9/00

(52) U.S. Cl. CPC A61K 36/534; A61K 36/07; A61K 9/0053

(57) ABSTRACT

Provided herein is a pharmaceutical composition and method for reducing intestinal pain and lessening foul-smelling stool and intestinal gas associated with large intestine disorders, comprising orally administering to a subject in need thereof an effective amount of the pharmaceutical composition comprising (i) champignon mushroom extract and (ii) peppermint or spearmint. The composition is formulated to provide controlled and/or delayed release of the active substances for specific efficacy in the large intestine.

### CROSS-REFERENCE TO RELATED APPLICATION

Priority is hereby claimed to provisional application Ser. No. 63/157,035, filed Mar. 5, 2021, which is incorporated herein by reference.

### BACKGROUND

The large intestine, also known as the large bowel, is the last part of the gastrointestinal tract and of the digestive system. It extracts water and salt from solid wastes before they are eliminated from the body and is the site in which flora-aided (largely bacterial) fermentation of unabsorbed material occurs.

Disorders of the large intestine adversely affect its function and may cause symptoms such as abdominal pain and cramps, excess gas, bloating, diarrhea, and constipation.

Presently, there are few dependable pharmaceutical treatment options for such disorders. For example, although there are treatment options including the use of bulking agents and more recently antibiotics and probiotics, such treatment options are not sufficiently effective. For example, bulking agents have not been shown to demonstrate an improvement in global symptoms and have been shown to increase bloating and pain (Tack et al. Aliment Pharmacol Ther. 2006 Jul. 15; 24(2):183-205). Antibiotic treatment is often challenged by the increasingly fast development of resistance of bacterial strains. Regarding probiotics, the magnitude of effect is uncertain due to inappropriate study design and not adequately reporting adverse events in clinical trials (Moayyedi et al. Gut. 2010 March;59(3):325-32; Brenner et al. Am J Gastroenterol. 2009 April;104(4):1033-49).

In view of the current shortcomings associated with treatments of large intestine disorders, there is a long-felt and unmet need for new methods and formulations to reduce bloating and odorous gas and feces.

### SUMMARY

Provided herein are pharmaceutical compositions and methods for reducing intestinal pain and lessening foul-smelling stool and intestinal gas associated with large intestine disorders.

The pharmaceutical composition provided herein comprises: (i) champignon extract, and (ii) peppermint and/or spearmint. The composition is suitable for oral administration with controlled and/or delayed release of the active substances for specific efficacy in the large intestine. In some embodiments, the pharmaceutical composition contains additional ingredients, including binders, fillers, desiccants, coloring agents, glidants, lubricants and flow regulating agents.

The champignon extract may be derived from any extraction of the mushroom Agaricus bisporus (champignon mushroom) suitably used for oral ingestion, in liquid or solid forms. The peppermint or spearmint may be prepared in any forms, including, but not limited to powders, crystals, extracts, and oils. The peppermint or spearmint may also be supplied as whole plant material, which may be fresh, dried, or freeze-dried (lyophilized). Alternatively, the plant material may be ground and/or compacted, or used in other forms known in the pharmaceutical arts.

Provided herein is also a method for reducing intestinal pain and lessening foul-smelling stool and intestinal gas associated with large intestine disorders, comprising orally administering to a subject in need thereof a pharmaceutical composition comprising (i) champignon extract and (ii) peppermint or spearmint, in an effective amount.

The pharmaceutical composition described herein may be administered to a subject at a dosage of about 60 mg to 10 g daily, which comprises between 10% to 90% of champignon extract, and between 10% to 90% of peppermint or spearmint, by weight of the total composition.

Thus, disclosed herein are the following:

1. An oral pharmaceutical composition for reducing intestinal pain and lessening foul-smelling stool and intestinal gas, comprising (i) champignon extract, and (ii) peppermint or spearmint,

wherein the champignon extract is present in the pharmaceutical composition in an amount between about 10% to about 90% by weight of the total composition, wherein the peppermint or spearmint is present in the pharmaceutical composition in an amount between about 10% to about 90% by weight of the total composition.

2. The pharmaceutical composition of claim 1, wherein the champignon extract is present in the pharmaceutical composition in an amount between about 20% to 80%, or between about 40% to 60%, by weight of the total composition.

3. The pharmaceutical composition of claim 1, wherein the peppermint or spearmint is present in the pharmaceutical composition in an amount between about 20% to 80%, or between about 40% to 60%, by weight of the total composition.

4. The pharmaceutical composition of claim 1, wherein the champignon extract is provided in liquid or solid forms.

5. The pharmaceutical composition of claim 1, wherein the peppermint or spearmint is provided in forms including powders, crystals, extracts and oils.

6. The pharmaceutical composition of claim 1, wherein the oral composition is formulated for oral administration to provide controlled release of the active substances for specific local effect in the large intestine.

7. The pharmaceutical composition of claim 1, wherein the oral composition is formulated for oral administration to provide delayed release of the active substances for specific local effect in the large intestine.

8. The pharmaceutical composition of claim 1, wherein the oral composition further comprises binders, fillers, desiccants, coloring agents, glidants, lubricants and flow regulating agents.

9. A method of reducing intestinal pain and lessening foul-smelling stool and intestinal gas, comprising orally administering to a subject in need thereof an effective amount of the pharmaceutical composition of claim 1.

10. The method of claim 7, wherein the amount administered to a subject is between 60 mg to 10 g daily.

11. The method of claim 7, wherein the amount administered to a subject is between 60 mg to 7.5 g, 60 mg to 5 g, or 60 mg to 2.5 g daily.

12. The method of claim 7, wherein the subject is a human.

### DETAILED DESCRIPTION

The following detailed description is presented to enable any person skilled in the pharmaceutical arts to make and use the composition of matter disclosed and claimed herein. For purposes of explanation, specific nomenclature is set forth to provide a thorough understanding of the composition. However, it will be apparent to one skilled in the art that these specific details are not required to make and use the composition. Descriptions of specific elements, ingredients, formulation methods, etc. are provided only as representative examples. The present application is not intended to be limited to the working examples and versions shown. Rather, the specification is to be accorded the widest possible scope consistent with the principles and features disclosed herein.

Unless otherwise defined, scientific and technical terms shall have the meanings that are commonly understood by those of ordinary skill in the pharmaceutical and/or nutraceutical art. Further, unless otherwise required by context, singular terms shall include pluralities and plural terms shall include the singular. That is, the indefinite articles “a” and “an” mean “one or more.”

As used herein the transitional term “comprising” and “comprises” are synonymous with “including,” “containing,” or “characterized by,” any one of which is inclusive or open-ended and does not exclude additional, unrecited elements or method steps, regardless of its use in the preamble or the body of a claim. The term further encompasses the terms “consisting of” and “consisting essentially of”. In the claims and/or the specification, “comprising” may mean “one,” but it is also consistent with the meaning of “one or more,” “at least one,” and “one or more than one.”

The term “delayed release” relates preferably to a pharmaceutical formulation that releases at least a portion of the active ingredients after a period of delay. In certain embodiments, the delay is sufficient for at least a portion of the active substances in the formulation to release in the large intestine.

The term “controlled release” refers preferably to a pharmaceutical formulation or component thereof that releases, or delivers, one or more active ingredients over a prolonged period of time. In certain embodiments, the period of time is sufficient for at least a portion of the active substances in a formulation to release in the large intestine.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the terms “prophylaxis” and “prevent” refer to delaying the onset of or reducing the likelihood of developing a disease or disorder or one or more symptoms thereof, as compared to an untreated control population.

An “effective amount” refers to a nontoxic but sufficient amount of a composition or material to provide a desired systemic or local effect. The effective amount will vary with the nature of the composition and constituent parts, the age and physical condition of the end user, the severity of the disease, the duration of the treatment, the nature of concurrent therapy, the particular pharmaceutically acceptable carrier utilized, and like factors. As used herein, all percentages are by weight unless otherwise specified.

Provided herein is a pharmaceutical composition comprising: (i) champignon extract, and (ii) peppermint or spearmint, for reducing intestinal pain and lessening foul-smelling stools and intestinal gas associated with large intestinal disorders, wherein the pharmaceutical composition is designed for a controlled and/or delayed release so that it releases (e.g. partially releases, selectively releases) in the large intestine.

Champignon extract derived from mushroom Agaricus bisporus is known to decrease levels of several toxic chemicals in the intestines and blood, and support the health and function of the kidneys (Nishihira et al. J Tradit Complement Med. 2015 Dec. 11;7(1):110-116; Bicak. Open Dent J. 2018 Apr. 30;12:322-330). Champignon extract is commercially available as Champex®-brand extract (Ricom Corporation, Japan; and U.S. distributor Maypro (NY)). Methods of preparing champignon extract suitably for oral ingestion have been available in the art. For example, hydrophilic solvent extracts of champignon mushroom are disclosed in U.S. Pat. Nos. 5,639,470 and 6,261,588, and European Patent No. 0 381 055. Other procedures for producing plant extracts (including hot extraction, cold extraction, and other techniques) may also be used. The liquid extracted from champignon mushrooms may be used in the pharmaceutical composition described herein. Alternatively, the extract may be dried (e.g. lyophilized) to form powders of the effective components.

Peppermint, also known as Mentha piperita, is a hybrid mint, a cross between watermint and spearmint. Peppermint has been extensively studied for treatment of intestinal disorders. A major constituent of peppermint is menthol, which blocks calcium channels in smooth muscle, thus producing antispasmodic effects on the gastrointestinal tract (Hawthorn et al. Aliment Pharmacol Ther. 1988 April;2(2):101-18). Peppermint possesses antimicrobial, anti-inflammatory, antioxidant, immunomodulating, and anesthetic activities (de Sousa Guedes et al. Int J Food Microbiol. 2016 Dec. 5;238:183-192; McKay and Blumberg. Phytother Res. 2006 August;20(8):619-33; Grigoleit. Phytomedicine. 2005 August;12(8):607-11). Spearmint oil is also shown to have anti-inflammatory activity (Direito et al. Medicines (Basel). 2019 Jun. 6;6(2):65).

Peppermint and spearmint are both commercially available in various forms, including powders (e.g., peppermint/spearmint leaf powder by Bixa Botanical (Mumbai, India), Starwest Botanicals (Sacramento, Calif., USA), and NutriCargo (Clifton, N.J., USA), crystals (e.g. peppermint crystals by Kotuku, Inc. (Cerritos, Calif., USA), extracts (e.g., peppermint/spearmint extracts by Starwest Botanicals, OliveNation (Avon, Mass., USA), and McCormick & Company (Hunt Valley, Md., USA), and oils (e.g. peppermint/spearmint oils by Starwest Botanicals, Nexon Botanics (San Diego, Calif., USA), NOW Essential Oils (Bloomingdale, Ill., USA), and OliveNation). Any of these forms or other forms of peppermint or spearmint may be used in the pharmaceutical compositions described herein.

The pharmaceutical composition may be formulated for oral administration as, for example but not limited to, powders, crystals, granules, small particles (which include particles sized on the order of micrometers, such as microspheres and microcapsules), particles (which include particles sized on the order of millimeters), beads, microbeads, pellets, pills, microtablets, compressed tablets, tablet triturates, molded tablets, and in capsules, which are either hard or soft and contain the composition as a powder, particle, bead, solution or suspension.

The pharmaceutical composition can also be formulated for oral administration as a solution or suspension in an aqueous liquid, as a liquid incorporated into a gel capsule or as any other convenient formulation for administration.

Preferably, the active substances, i.e. champignon extract and peppermint or spearmint, are used in a pharmaceutical formulation that protects the largest possible amount of active substance from being absorbed by the body in the upper small intestine and rather effects a release (e.g., controlled release and/or delayed release) into the large intestine (e.g., at least a portion of the active substances are selectively released in the large intestine). For example, the controlled and/or delayed release formulation may be formulated to release substantially the entire active substances from the dosage form during passage through the large intestine. As a result, release of the active substances in the stomach and small intestine can be substantially avoided. In some embodiments, the controlled and/or delayed release formulation may be formulated to release greater than 50% of the active substances in the large intestine, such as a release of the active substances in the large intestine that is greater than about 55%, 60%, 65%, 70%, 75%, 80%, and 90%. In another embodiment, the controlled and/or delayed release formulation is formulated to release from about 60 to 75% of the active substances in the large intestine.

Examples of controlled release and/or delayed release formulations designed for releasing active substances in the large intestine are known in the art. See, for example, U.S. Pat. Nos. 5,217,720; 5,283,064; 5,866,619; 5,900,252; 6,248,362; 6,368,629; 8,747,893; and 9,138,430.

The retardation of release of the active ingredients is advantageously achieved, e.g., by coatings which are resistant to gastric juice and dissolve depending on the pH, by means of microcellulose and/or multimatrix (MMX) technologies, by using different carrier matrices or a combination of these techniques. Examples include film coatings which contain acrylic and/or methacrylate polymers in various mixtures for controlled and/or delayed release. For example, the active substance(s) can be contained in a conventional matrix of microcrystalline cellulose or gelatin or with multimatrix technology, which is coated with a material, which provides the delayed release of the active substance(s).

It is preferred to introduce an active substance in capsules which are coated by means of known methods. Suitable coating agents include, for example, pharmaceutically usable polymeric substances, such as EUDRAGIT RETARD®-brand capsules (a copolymer of ethyl methacrylate and trimethylacrylethylammonium chloride, Evonik Operations GmbH, Essen, Germany), EUDRAGIT L®-brand capsules (a copolymer of methyl methacrylate and methacrylate), EUDRAGIT S®-brand capsules (a copolymer of methyl methacrylate and methacrylate), ETHOCEL®-brand capsules (ethylcellulose, The Dow Chemical Corporation, Midland, Mich., USA), METOLOSE®-brand capsules (methylcellulose, Shin-Etsu Chemical Co., Ltd., Tokyo, Japan), HPMCP®-brand capsules (hydroxypropylmethylcellulose phthalate, Shinetsu Chemical Co., Ltd.), and AQOAT®-brand capsules (hydroxypropylmethylcellulose acetate succinate, Shinetsu) as a cellulose derivative, and the mixtures thereof. Where appropriate, water soluble polymers (e.g., polyvinylpyrrolidone), water-soluble celluloses (e.g., hydroxypropyl methylcellulose (HPMC) or hydroxypropyl cellulose (HPC)), polysorbate 80, polyethylene glycol (PEG), lactose or mannitol can also be contained in the coating material.

The volume of capsules may be 0.13, 0.21, 0.30, 0.37, 0.50, 0.68, 0.95, and 1.37 ml, with locked length of 11.10, 14.30, 15.90, 18.00, 19.40, 21.70, 23.30, and 26.14 mm (+/−0.76 mm) respectively. The average weight of capsules may be 28, 38, 48, 61, 76, 96, 118, and 163 mg (+/− 10%), which can fill ingredients ranging from 60 to 1370 mg.

The pharmaceutical composition provided herein may further contain other pharmaceutical excipient substances, such as binders, fillers, desiccants, coloring agents, glidants, lubricants and flow regulating agents. Binders, such as but not limited to, starch, gelatin, sucrose, glucose, dextrose, molasses, lactose, acacia gum, sodium alginate, extract of Irish moss, panwar gum, ghatti gum, mucilage of isapgol husks, carboxymethylcellulose, methylcellulose, polyvinylpyrrolidone, Veegum and starch arabogalactan, polyethylene glycol, ethylcellulose, and waxes, may be added to the pharmaceutical composition to increase its cohesive qualities. Fillers, such as dextrates, dicalcium phosphate, calcium sulfate, lactose, cellulose, kaolin, mannitol, sodium chloride, dry starch, sorbitol, sucrose, inositol, powdered sugar, bentonite, microcrystalline cellulose, or hydroxypropylmethylcellulose may be added to increase the bulk of the composition. Also, glidants, such as but not limited to, colloidal silicon dioxide or talc may be added to improve the flow characteristics of a powder. Additionally, lubricants, such as but not limited to, talc, magnesium stearate, calcium stearate, stearic acid, hydrogenated vegetable oils, polyethylene glycol, sodium benzoate, sodium acetate, sodium chloride, leucine, carbowax, sodium lauryl sulfate, and magnesium lauryl sulfate may be added.

The pharmaceutical composition can be formulated, where appropriate, together with further active substances and with excipients conventional in pharmaceutical compositions, e.g., talcum, gum arabic, lactose, starch, magnesium stearate, cocoa butter, aqueous and non-aqueous carriers, lipid components of animal or vegetable origin, paraffin derivatives, glycols (in particular polyethylene glycol), various plasticizers, dispersants, emulsifiers and/or preservatives.

Also provided herein is a method of reducing intestinal pain and lessening foul-smelling stool and intestinal gas associated with large intestine disorders, comprising orally administering to a subject in need thereof a pharmaceutical composition comprising (i) champignon extract and (ii) peppermint or spearmint, in an effective amount. The method is equally usable for the therapy or prophylaxis of diseases with similar genesis. The subject may be any type of mammal, but is preferably a human.

In some embodiment, dosage forms of the pharmaceutical composition contain champignon extract in an amount between about 10% to 90%, between about 20% to 80%, or between about 40% to 60% by weight of the total composition; and contain peppermint or spearmint in an amount between about 10% to 90%, between about 20% to 80%, or between about 40% to 60% by weight of the total composition.

In one embodiment, the amount of dosage forms administered to a subject is between 60 mg to 10 g daily. In some embodiments, the amount of dosage forms administered to a subject is between about 60 mg to 10 g, 60 mg to 7.5 g, 60 mg. to 5 g, and 60 mg to 2.5 g daily. Depending on the intensity and severity of the disease, the dosage forms are administered once or several times daily, for example 2, 3, or 4 times daily, or in another dosage regimen to be chosen by a physician.

What is claimed is:

1. An oral pharmaceutical composition for reducing intestinal pain and lessening foul-smelling stool and intestinal gas, comprising (i) champignon extract, and (ii) peppermint or spearmint,
wherein the champignon extract is present in the pharmaceutical composition in an amount between about 10% to about 90% by weight of the total composition,
wherein the peppermint or spearmint is present in the pharmaceutical composition in an amount between about 10% to about 90% by weight of the total composition.

2. The pharmaceutical composition of claim 1, wherein the champignon extract is present in the pharmaceutical composition in an amount between about 20% to 80%, or between about 40% to 60%, by weight of the total composition.

3. The pharmaceutical composition of claim 1, wherein the peppermint or spearmint is present in the pharmaceutical composition in an amount between about 20% to 80%, or between about 40% to 60%, by weight of the total composition.

4. The pharmaceutical composition of claim 1, wherein the champignon extract is provided in liquid or solid forms.

5. The pharmaceutical composition of claim 1, wherein the peppermint or spearmint is provided in forms including powders, crystals, extracts and oils.

6. The pharmaceutical composition of claim 1, wherein the oral composition is formulated for oral administration to provide controlled release of the active substances for specific local effect in the large intestine.

7. The pharmaceutical composition of claim 1, wherein the oral composition is formulated for oral administration to provide delayed release of the active substances for specific local effect in the large intestine.

8. The pharmaceutical composition of claim 1, wherein the oral composition further comprises binders, fillers, desiccants, coloring agents, glidants, lubricants and flow regulating agents.

9. A method of reducing intestinal pain and lessening foul-smelling stool and intestinal gas, comprising orally administering to a subject in need thereof an effective amount of the pharmaceutical composition of claim 1.

10. The method of claim 7, wherein the amount administered to a subject is between 60 mg to 10 g daily.

11. The method of claim 7, wherein the amount administered to a subject is between 60 mg to 7.5 g, 60 mg to 5 g, or 60 mg to 2.5 g daily.

12. The method of claim 7, wherein the subject is a human.
